(19) United States
(12) Patent Application Publication
(10) Pub. No.: US 2008/0109047 A1
(43) Pub. Date: May 8, 2008

(54) Apnea treatment device

(76) Inventor: Benjamin David Pless, Atherton, CA (US)

Correspondence Address: RMX, L.L.C., P.O. Box 3550 Loyola Corners Station, Los Altos, CA, 94024, US

(21) Appl. No.: 11/977,087

(22) Filed: Oct. 23, 2007

Related U.S. Application Data
(60) Provisional application No. 60/854,616, filed on Oct. 26, 2006.

(51) Int. Cl. A61N 1/18

(52) U.S. Cl. 607/42

(57) ABSTRACT

Treatment or control of sleep apnea by achieved using a device or method for stimulation of expiration muscles. Somatic or expiratory muscle stimulation instead of a mask during sleep may regularize breathing. An apnea belt around the thorax may detect respiration by monitoring stretch and provide electrical stimulation to muscles used for expiration.

### FIELD OF THE INVENTION

This invention relates to treatment or control of sleep apnea by stimulation of expiration muscles.

### BACKGROUND OF THE INVENTION

This invention is directed to the treatment or control of sleep apnea by stimulation of expiration muscles. Other techniques reported to control sleep apnea include continuous positive airway pressure (for example U.S. Pat. No. 7,004,808), hypoglossal nerve stimulation (for example U.S. Pat. No. 6,587,725), upper airway stimulation (for example U.S. Pat. No. 6,770,022), and diaphragm stimulation (for example U.S. Pat. No. 5,146,918). None of these references recognizes the beneficial possibility of stimulation of expiratory muscles. Jurji Sorli (“Ventilatory Assist Using Electrical Stimulation of Abdominal Muscles”, IEEE Transactions of Rehabilitation Engineering, Vol. 4, No. 1, March 1996) provides observations on the effect of abdominal stimulation but does not recognize the value of stimulating expiratory muscles to control sleep apnea.

### SUMMARY OF THE INVENTION

Conventional sleep apnea therapy uses a technique known as CPAP (continuous positive airway pressure). CPAP is effective in controlling apnea, but since it requires that patients wear a tight fitting pressurized mask while sleeping it is often a difficult therapy for patients to comply with the therapy on a consistent basis because of discomfort. The invention that is the subject of the disclosure uses somatic or expiratory muscle stimulation instead of a mask during sleep to regularize breathing. In one embodiment, an apnea belt around the thorax detects respiration by monitoring stretch and provides electrical stimulation to muscles used for expiration. These muscles include, without limitation, abdominal muscles (including the transverse abdominals), internal oblique muscles, external oblique muscles, intracostal muscles and scalene muscles. The stimulation may be synchronized to the expiration phase of the breathing cycle and may be applied with every breath, every other breath, or less frequently. By monitoring stretch, the apnea belt can keep track of inspiration rate and regularity and can use algorithms to help the patient achieve therapeutically significant inspiration and regularity targets.

In the simplest embodiment, the invention provides electrical stimulation (on a pre-set basis) to expiratory or somatic muscles in a manner intended to result in the patient's respiration synchronizing (at least in part) with the applied stimulation.

### BRIEF DESCRIPTION OF THE DRAWINGS

FIG. 1 shows a diagram of a simple embodiment of the apnea control device.

FIG. 2 shows a stimulation pattern used by the apnea control device.

FIG. 3 shows a diagram of a further embodiment of the apnea control device.

FIG. 4 shows the elements of the circuit operation of the further embodiment of the apnea control device.

### DETAILED DESCRIPTION OF THE INVENTION

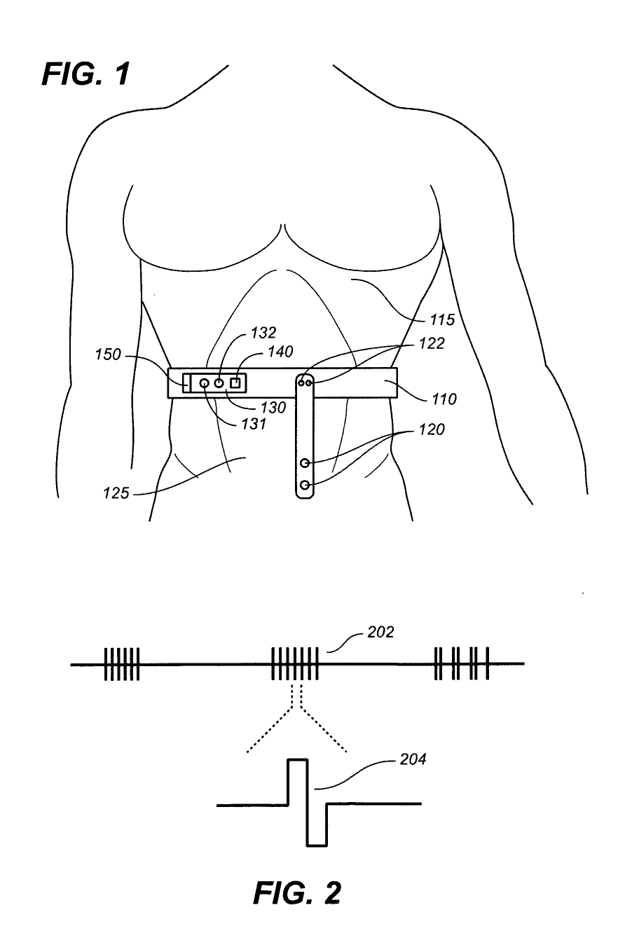

A simple embodiment of the invention is shown if FIG. 1. A belt 110 is affixed around a patient's torso 115. Electrodes 120 extend down to the patient's abdomen 125, and are secured over one or more of the motor points of the abdominal muscles. The electrodes 120 may be of a number of different technologies including metal foil requiring that the patient apply gel, or may be pre-gelled, or may be percutaneous. The electrodes 120 may be disposable, and may be connected to the belt 110 by electrically conductive snaps 122. The electrodes may be secured to the patient by a number of techniques including adhesive, tape, or the compression supplied by the belt 110. The belt 110 includes a control panel 130. The control panel 130 has rotary switches (131, 132) to allow adjustment of the stimulation rate and intensity respectively. The intensity may be set to “zero” to give the patient the opportunity to turn the device off. In the embodiment shown, the rotary switches 131, 132 are adjustable with a coin or screwdriver to reduce the possibility that the selected settings might be inadvertently changed due to movement while the patient sleeps. The control panel 130, includes a timer to delay the beginning of stimulation for a period of time (for example 30 minutes) to give the patient time to fall asleep before stimulation starts. The timer begins timing when the amplitude setting 131 is set to a value other than “zero”. The control panel 130 contains a circuit 140 that produces the electrical pulses that are conducted to the electrodes 120. The circuit 140 is powered by batteries 150. The batteries 150 may be replaceable, rechargeable, or the belt 110 may be disposed of when the batteries are depleted.

This is a single embodiment of a simple implementation of the invention. Augmentations including adjustability of the pulse width of the stimulation pulses, adjustability of the delay timer, the ability to connect the belt to a computer to make adjustments, a low battery indicator, and the ability of the patient to turn on stimulation to adjust the amplitude are also anticipated. Furthermore it is anticipated that instead of a belt 110, a vest or adhesive patches may be used for the same purpose.

FIG. 2 shows an example of a stimulation pattern applied to the patient. A burst of pulses on the order of 500 msec in duration 202, is composed of individual pulses 204, which have a pulse-to-pulse intervals of about 20 milliseconds 206. The individual pulses 204 are biphasic and are about 250 microseconds in duration, and have an amplitude that can be set by the amplitude rotary switch 132. A typical range for the pulse amplitude is 1 to 100 milliamperes. The interval between bursts 202 is set by the stimulation rate rotary switch 131. A typical range for the interval is one to ten seconds.

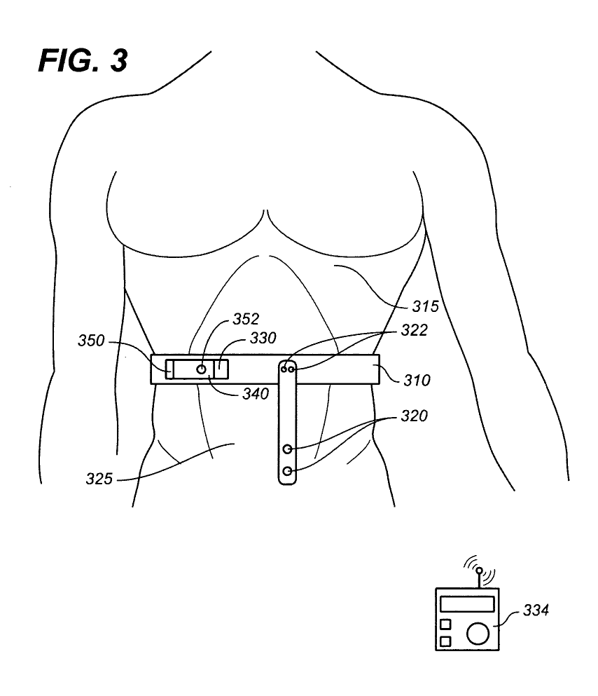

FIG. 3 provides a further embodiment of the invention. A belt 310 is affixed around a patient's torso 315. Electrodes 320 extend down to the patient's abdomen 325, and are secured over one or more of the motor points of the abdominal muscles. The electrodes 320 may be of a number of different technologies including metal foil requiring that the patient apply gel, or may be pre-gelled, or may be percutaneous. The electrodes 320 may be disposable, and are connected to the belt 310 by electrically conductive snaps 322. The electrodes may be secured to the patient by a number of techniques including adhesive, tape, or the compression supplied b the belt 310. The belt 310 includes a wireless interface 330. The wireless interface 330 allows the belt to communicate with a separate controller 334. The controller 334 is used to set different parameters regarding the performance of the belt. A circuit 340 is powered by batteries 350. The batteries 350 may be replaceable, rechargeable, or the belt 310 may be disposed of when the batteries are depleted. Preferably the batteries 350 are coin cells that can be easily replaced by the patient. A rotary switch 352 is used by the patient to turn the apnea control device on and off.

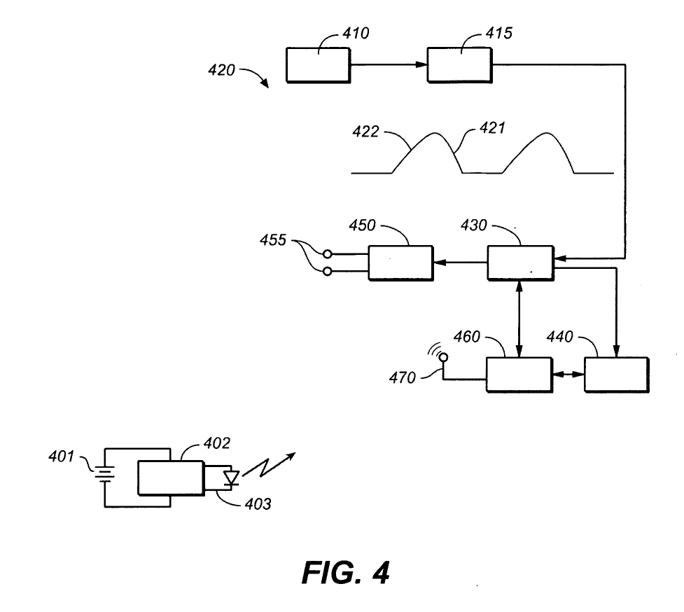

The elements of the circuit 340 are shown in the diagram of FIG. 4. Power is supplied by batteries 401. A power management circuit 403 provides regulated power for the other circuit elements and controls an indicator for low battery 403, in this case shown as an LED. A respiration detector 410 may be a stretch transducer (for example in a belt) or may detect respiration through motion, plethysmography, or other techniques. The output of the respiration detector 410 is processed by a signal conditioner 415 that includes filtering and analog to digital conversion. The conditioned respiration signal 420 indicates inspiration 422 and exhalation 421. In this figure the respiration signal 420 is shown as a continuous time signal for clarity; in fact it is a digital signal that can be interpreted by the microprocessor 430. The microprocessor 430 analyzes the respiration signal 422 and determines when and what stimulation to apply to the patient through the electrodes 455. At the appropriate time, generally in the middle of the exhalation 421, the microprocessor commands the stimulator 450 to deliver stimulation to the patient through electrodes 455. The stimulator 450 delivers electricity in a form suitable to stimulate the selected patient muscle of muscles. Typically the stimulation is in the form of pulses as shown in FIG. 2.

Note that the microprocessor 430 can store diagnostic and respiration waveforms and information in a storage device 440. The storage device 440 may be flash memory, a hard drive, static RAM or other storage medium. The information stored in the storage device 440 may be uploaded to a separate controller for review by a clinician to assess the functioning of the apnea control device and the status of the patient. In addition, it is anticipated that the apnea control device could have electrodes to detect ECG and that the ECG information could be stored along with the respiration information.

Communication from a separate controller device with the apnea control device can occur wirelessly through the action of the antenna 470 and the communication link manager 460. Wireless communication may be through a cell phone data link, Bluetooth, the MISC band, hospital telemetry band or other suitable wireless frequency. In the alternative, infrared or other optical communication means may be used. While the preferred embodiment is wireless, the use of a cable hook-up for communication between the apnea control device and a separate controller is also anticipated.

In the preferred embodiment, the microprocessor automatically determines the correct amplitude setting for the stimulator 450 to deliver pulses. The microprocessor can accomplish automatic amplitude setting by running an algorithm. The algorithm collects information regarding the patient's respiration rate, regularity and pattern in the absence of stimulation. The microprocessor begins stimulation at a low amplitude setting, and applies the stimulation during exhalation 421. If there is no change in the patient's respiration rate, regularity or pattern, the stimulation amplitude is increased until the microprocessor identifies a change as a result of stimulation. The lowest stimulation amplitude that produces a consistent change is automatically selected by the microprocessor for subsequent stimulations. Verifying the correct setting can occur automatically at predetermined intervals, or if the microprocessor determines that the current setting is no longer effective.

Note that while the embodiments shown have been directed to external devices to control sleep apnea, it is anticipated that an implantable version would also be useful and could operate by similar principles: detection of respiration and stimulation of one (or more) expiration muscle(s) at a time other than during inspiration. Furthermore, the embodiments shown have disclosed electrical stimulation, but it is anticipated that laser, microwave and vibrational energy could also be employed.

What is claimed is:

1. A device for treating apnea comprising:
a. electrodes,
b. electrical stimulator electrically connected to said electrodes,
c. wherein at least one electrode is positioned to cause stimulation of a least one expiratory muscle.
